(19) United States
(12) Patent Application Publication
(10) Pub. No.: US 2023/0001159 A1
(43) Pub. Date: Jan. 5, 2023

(54) Vascular Access Device to Dispense Blood for Point-Of-Care Testing

(71) Applicant: Becton, Dickinson and Company, Franklin Lakes, NJ (US)

(72) Inventors: John Lackey, West Valley City, UT (US); Jonathan Karl Burkholz, Salt Lake City, UT (US); Weston F. Harding, Lehi, UT (US); Megan Scherich, Salt Lake City, UT (US)

(21) Appl. No.: 17/852,538

(22) Filed: Jun. 29, 2022

Related U.S. Application Data
(60) Provisional application No. 63/218,157, filed on Jul. 2, 2021.

(51) Int. Cl. A61M 25/09; A61M 39/02

(52) U.S. Cl. CPC A61M 25/09; A61M 39/0247; A61M 2039/0258; A61M 2039/0202

(57) ABSTRACT

An instrument advancement device may include a housing, which may include a distal end and a proximal end. An instrument, such as a vascular access instrument configured to enter vasculature of a patient, may be disposed within the housing. The instrument advancement device may include an advancement element. In response to movement of the advancement element with respect to the housing, the instrument may be configured to advance distal to the distal end of the housing. The housing or a distal end of the housing may include a compressible portion proximate a chamber configured to hold blood. In response to compression of the compressible portion, the housing may be configured to dispense blood distally from the chamber through the distal end of the housing.

### CROSS-REFERENCE TO RELATED APPLICATION

The present application claims priority to U.S. Provisional Application Ser. No. 63/218,157, entitled “Vascular Access Device to Dispense Blood for Point-Of-Care Testing”, filed Jul. 2, 2021, the entire disclosure of which is hereby incorporated by reference in its entirety.

### BACKGROUND OF THE INVENTION

Catheters are commonly used for a variety of infusion therapies. For example, catheters may be used for infusing fluids, such as normal saline solution, various medicaments, and total parenteral nutrition, into a patient. Catheters may also be used for withdrawing blood from the patient.

A common type of catheter device includes a catheter that is over-the-needle. As its name implies, the catheter that is over-the-needle may be mounted over an introducer needle having a sharp distal tip. A catheter assembly may include a catheter adapter, the catheter extending distally from the catheter adapter, and the introducer needle extending through the catheter. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of the catheter assembly. Once placement of the needle has been confirmed, the clinician may temporarily occlude flow in the vasculature and remove the needle, leaving the catheter in place for future blood withdrawal or fluid infusion.

Infusion and blood withdrawal using the catheter may be difficult for several reasons, particularly when an indwelling time of the catheter increase. A fibrin sheath or thrombus may form on an internal surface of the catheter assembly, an external surface of the catheter assembly, or within the vasculature near the distal tip of the catheter. The fibrin sheath or thrombus may block or narrow a fluid pathway through the catheter, which may impair infusion and/or collection of a high-quality blood sample.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure relates generally to vascular access devices and related systems and methods. In some embodiments, an instrument advancement device may include a housing, which may include a distal end and a proximal end. In some embodiments, the instrument advancement device may include an instrument disposed within the housing. In some embodiments, the instrument advancement device may include an advancement element. In some embodiments, in response to movement of the advancement element with respect to the housing, the instrument may be configured to advance distal to the distal end of the housing.

In some embodiments, the distal end of the housing may include a compressible portion proximate a chamber configured to hold blood. In some embodiments, in response to compression of the compressible portion, the housing may be configured to dispense blood distally from the chamber through the distal end of the housing. In some embodiments, in response to compression of the compressible portion, the housing may be configured to dispense a small amount of blood that may be used for point-of-care testing.

In some embodiments, the instrument may include a guidewire. In some embodiments, the vascular advancement device may include another instrument coupled to the advancement element. In some embodiments, the other instrument may include a tubing. In some embodiments, blood may be configured to flow proximally through the tube. In some embodiments, the guidewire may be disposed within the tubing.

In some embodiments, the distal end of the housing may include a distal connector, and the distal connector may include the compressible portion and the chamber. In some embodiments, the instrument advancement device may include a tab, which may form an upper surface of the compressible portion. In some embodiments, the distal connector may include a pair of opposing lever arms, and the tab may be disposed between the pair of opposing lever arms.

In some embodiments, the housing may include a slot. In some embodiments, the advancement element may extend through the slot and may be configured to move linearly along the slot between a retracted position and an advanced position. In some embodiments, in response to movement of the advancement element from the retracted position to the advanced position, the instrument may be configured to advance distal to the distal end of the housing.

In some embodiments, the housing may include the slot, and the advancement element may extend through the slot and may be configured to move linearly along the slot. In some embodiments, the instrument may include the guidewire, which may include a first end and a second end. In some embodiments, in response to movement of the advancement element distally a first distance along the slot, the second end of the guidewire may be configured to advance a second distance. In some embodiments, the second distance may be at least twice the first distance. In some embodiments, the instrument advancement device may include the other instrument, which may include the tubing, which may include a distal end and a proximal end. In some embodiments, in response to movement of the advancement element distally the first distance along the slot, the proximal end of the tubing may be configured to advance the first distance.

In some embodiments, the housing may include an extension tube extending through the advancement element. In some embodiments, the instrument advancement device may include a wedge disposed within the advancement element and the extension tube. In some embodiments, a pair of opposing pinch members may be configured to pinch the extension tube. In some embodiments, the pair of opposing pinch members may be disposed within the advancement element and configured to move along the extension tube with the advancement element. In some embodiments, the instrument may extend distally from the wedge. In some embodiments, in response to moving the advancement element distally along the extension tube, the pair of opposing pinch members may push the wedge distally, and the instrument may be configured to advance distal to the distal end of the housing.

In some embodiments, the instrument advancement device may include the housing, which may include the proximal end, the distal end, and the slot. In some embodiments, the advancement element may extend through the slot and may be configured to move linearly along the slot between the retracted position and the advanced position. In some embodiments, in response to movement of the advancement element linearly along the slot from the retracted position to the advanced position, the instrument may be advanced beyond the distal end of the housing. In some embodiments, the housing may include a compressible portion, and in response to compression of the compressible portion, the housing may be configured to dispense blood distally through the distal end of the housing.

In some embodiments, the instrument may include the guidewire. In some embodiments, the housing may include a lumen configured for blood to flow therethrough, and the compressible portion may be proximate the lumen. In some embodiments, the proximal end of the housing may include a proximal connector. In some embodiments, the instrument advancement device may include an extension tube extending through the lumen and coupled to the proximal connector. In some embodiments, the blood may be configured to flow through the extension tube. In some embodiments, in response to compression of the compressible portion, the extension tube may be pinched, and the housing may be configured to dispense the blood from the extension tube distally through the distal end of the housing. In some embodiments, the blood may include a small volume to be used for point-of-care testing. In some embodiments, the compressible portion may be aligned with the slot.

In some embodiments, the distal end of the housing may include a slot and a slider configured to move along the slot. In some embodiments, in response to movement of the slider from a proximal end of the slot to a distal end of the slot, blood may be dispensed distally through the distal end of the housing. In some embodiments, the blood may include a small volume to be used for point-of-care testing. In some embodiments, the slider may be coupled to a septum disposed within a chamber of the distal end of the housing. In some embodiments, the septum may be configured to move with the slider. In some embodiments, the distal end of the housing may include a chamber configured to hold blood, and the septum may extend across the chamber. In some embodiments, the instrument may include the guidewire.

In some embodiments, the distal end of the housing may include a distal connector, and the distal connector may include the slider, the slot, and the septum. In some embodiments, the distal connector may include a pair of opposing lever arms. In some embodiments, the slider may be disposed between the pair of opposing lever arms.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1A is an upper perspective view of an example instrument advancement device, illustrating an example advancement element in an example initial or retracted position, according to some embodiments;

FIG. 1B is a longitudinal cross-sectional view of the instrument advancement device of FIG. 1A;

FIG. 1C is a longitudinal cross-sectional view of the instrument advancement device of FIG. 1A, illustrating the advancement element in the initial or retracted position and an example compressible portion being compressed, according to some embodiments;

FIG. 1D is a cross-sectional view of the instrument advancement device of FIG. 1A along the line 1D-1D of FIG. 1A, according to some embodiments;

FIG. 1E is an enlarged view of a portion of FIG. 1D, according to some embodiments;

FIG. 1F is a cross-sectional view of the instrument advancement device of FIG. 1A along the line 1F-1F of FIG. 1A, according to some embodiments;

FIG. 2A is an upper perspective view of the instrument advancement device, illustrating the advancement element in the initial or retracted position, according to some embodiments;

FIG. 2B is a cross-sectional view of the instrument advancement device of FIG. 2A;

FIG. 2C is an enlarged cross-sectional view of a portion of the instrument advancement device of FIG. 2A, according to some embodiments;

FIG. 2D is a cross-sectional view of the instrument advancement device of FIG. 2A along the line 2D-2D of FIG. 2A, according to some embodiments;

FIG. 2E is an enlarged view of a portion of FIG. 2D, according to some embodiments;

FIG. 2F is an upper perspective view of the instrument advancement device of FIG. 2A coupled to an example catheter assembly, illustrating the advancement element in an example advanced position, according to some embodiments;

FIG. 3A is an upper perspective view of an example instrument advancement device, according to some embodiments;

FIG. 3B is a cross-sectional view of an example distal end of the instrument advancement device of FIG. 3A, according to some embodiments;

FIG. 3C is a cross-sectional view of another example distal end of the instrument advancement device of FIG. 3A, according to some embodiments;

FIG. 3D is a cross-sectional view of an example advancement element of the instrument advancement device, illustrating an example extension tube extending therethrough, according to some embodiments;

FIG. 3E is an upper perspective view of an example piece of the advancement element, according to some embodiments;

FIG. 3F is an upper perspective view of another example piece of the advancement element configured to couple to the piece of the housing of FIG. 3E, according to some embodiments;

FIG. 4A is a cross-sectional side view of an example instrument advancement device, according to some embodiments;

FIG. 4B is an exploded rear view of an example instrument advancement mechanism of the instrument advancement device of FIG. 4A;

FIG. 5A is an upper perspective view of an example instrument advancement device, according to some embodiments;

FIG. 5B is an enlarged upper perspective view of a portion of the instrument advancement device of FIG. 5A, according to some embodiments;

FIG. 5C is a cross-sectional view of the instrument advancement device of FIG. 5A along the line 5C-5C of FIG. 5A, with an example advancement element removed for illustrative purposes, according to some embodiments;

FIG. 5D is a cross-sectional view of the instrument advancement device of FIG. 5A along the line 5D-5D of FIG. 5A, according to some embodiments;

FIG. 5E is a lower perspective view of the instrument advancement device of FIG. 5A, according to some embodiments;

FIG. 6A is a cross-sectional view of an example distal end of an example instrument advancement device, illustrating an example slider in an example retracted position, according to some embodiments; and

FIG. 6B is a cross-sectional view of the distal end of the instrument advancement device of FIG. 6A, illustrating the slider in an example advanced position, according to some embodiments.

### DETAILED DESCRIPTION

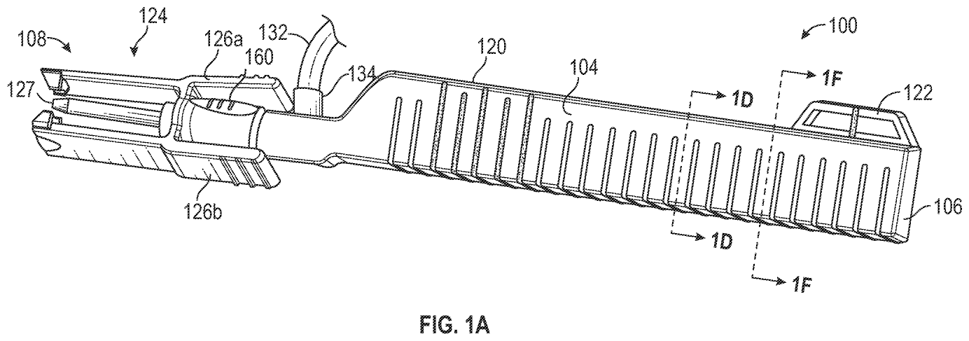

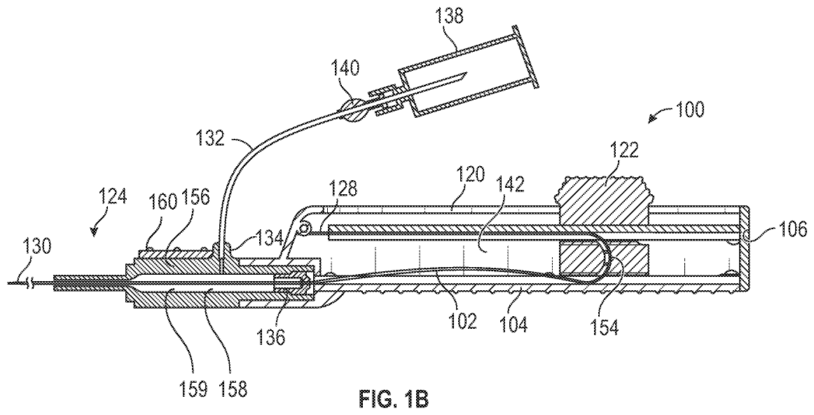

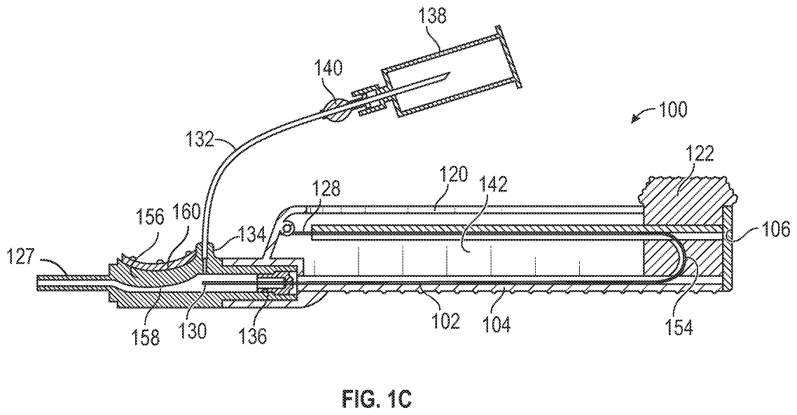

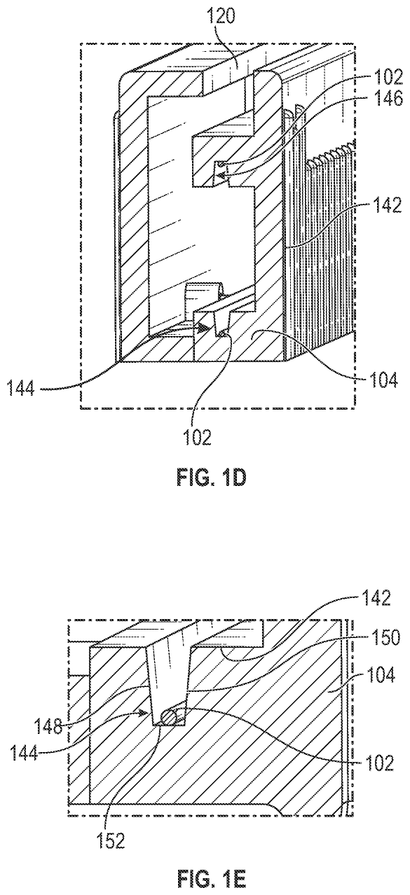

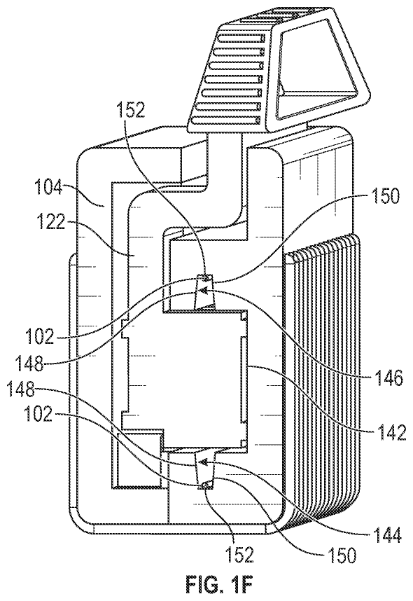

Referring now to FIGS. 1A-1F, in some embodiments, an instrument advancement device 100 may be configured to deliver an instrument 102 through a catheter of a catheter assembly. In some embodiments, the instrument 102 may be advanced through the catheter to push past any occlusions in the catheter or vasculature (e.g., thrombus or fibrin sheath at a tip of the catheter, vein collapse, valves, etc.) to create a clear pathway for fluid flow. In some embodiments, the instrument 102 may reduce or remove occlusions, improving patency of the catheter for medication and fluid delivery, as well as blood acquisition, during a dwell time of the catheter.

In some embodiments, the instrument 102 may include a guidewire, a probe, a guidewire or a probe with one or more sensors, or another suitable instrument. In some embodiments, the sensors may be used for patient or device monitoring and may include sensors measuring pressure, temperature, pH, blood chemistry, oxygen saturation, flow rate, or another physiological property. In some embodiments, the catheter may include a peripheral IV catheter, a peripherally-inserted central catheter, or a midline catheter. In some embodiments, the catheter through which the instrument 102 may be delivered may have been previously inserted into vasculature of a patient and may be dwelling within the vasculature when the instrument 102 is advanced through the catheter.

In some embodiments, the instrument 102 may be disposed within a housing 104, which may be configured to protect the instrument 102 from damage and/or contamination from a surrounding external environment. In some embodiments, the housing 104 may be rigid or semi-rigid. In some embodiments, the housing 104 may be made of one or more of stainless steel, aluminum, polycarbonate, metal, ceramic, plastic, and another suitable material. In some embodiments, the housing 104 may include a proximal end 106, a distal end 108, and a slot 120. In some embodiments, the slot 120 may extend parallel to a longitudinal axis of the housing 104.

In some embodiments, the instrument advancement device 100 may include an advancement element 122, which may extend through the slot 120 and may be configured to move linearly along the slot 120 between a retracted position illustrated, for example, in FIG. 1A, and an advanced position. In some embodiments, the user may pinch or grasp the advancement element 122 to move the advancement element 122 between the retracted position and the advanced position.

In some embodiments, the distal end 108 of the housing 104 may include a distal connector 124. In some embodiments, the distal connector 124 may include opposing lever arms 126a,126b. In some embodiments, distal ends of the opposing lever arms 126a,126b may be configured to move apart from each other in response to pressure applied to proximal ends of the opposing lever arms 126a,126b. In some embodiments, in response to removal of the pressure applied to the proximal ends of the opposing lever arms 126a,126b, the distal ends may move closer to each other and clasp a portion of the catheter assembly, such as a needleless connector, another connector, or a proximal end of a catheter adapter, for example. In some embodiments, the distal connector 124 may include a blunt cannula 127 or a male luer configured to insert into the portion of the catheter assembly.

In some embodiments, the distal connector 124 may include any suitable connector. For example, the distal connector 124 may include a threaded male luer, a slip male luer, a threaded male luer with a spin lock, a threaded male luer with a removable blunt cannula snap connection, a slip male luer with a removable blunt cannula snap connection, or another suitable connector. In some embodiments, the distal connector 124 may include one or more bond pockets, which may each be configured to receive an extension tube. In some embodiments, the distal connector 124 may be monolithically formed as a single unit with a body of the housing 104 that includes the slot 120.

In some embodiments, the instrument 102 may include a first end 128 and a second end 130. In some embodiments, movement of the advancement element 122 from the retracted position to the advanced position may cause the second end 130 of the instrument 102 to be advanced beyond the distal end 108 of the housing 104. In some embodiments, moving the advancement element 122 to the advanced position may introduce the instrument 102 into the catheter assembly and/or through the catheter. In some embodiments, in response to the instrument 102 being introduced into the catheter assembly and/or through the catheter, the instrument 102 may access a fluid pathway of the catheter assembly and/or the vasculature of a patient.

In some embodiments, the catheter of the catheter assembly with significant dwelling time within the vasculature may be susceptible to narrowing, collapse, kinking, blockage by debris (e.g., fibrin or platelet clots), and adhering of a tip of the catheter to the vasculature. Thus, blood withdrawal using the catheter may be difficult. In some embodiments, the instrument 102 may have a diameter less than a diameter of the catheter of the catheter assembly to provide access to the vasculature of the patient without any additional needle sticks. In some embodiments, the instrument 102 may clear the pathway for collecting a blood sample. Thus, in some embodiments, the instrument advancement device 100 may be used for needle-free blood collection and/or fluid infusion.

In some embodiments, an extension tube 132 may be coupled to the instrument advancement device 100, and the extension tube 132 may be used for blood collection and/or fluid infusion. In some embodiments, the extension tube 132 may extend from a port 134 of the housing 104. In some embodiments, a septum 136 may be within the housing 104 to enable the instrument 102 to advance and/or retract while maintaining a closed fluid path. In some embodiments, the instrument 102 may be configured to extend through the septum 136. In some embodiments, the septum 136 may be disposed proximal to the port 134 and distal to the advancement element 122 in the advanced position. In some embodiments, the septum 136 may include silicone, rubber, an elastomer, or another suitable material. In some embodiments, the septum 136 may include an aperture, slit, or the like to accommodate the instrument 102 therethrough.

In some embodiments, a proximal end of the extension tube 132 may be coupled to a blood collection device 138. For example, the proximal end of the extension tube 132 may be integrated with a connector 140, which may be coupled to the blood collection device 138. In some embodiments, a needleless connector may be disposed between the connector 140 and the blood collection device 138. In some embodiments, the connector 140 and/or the port 134 may be coupled to an IV line or another fluidic connection.

In some embodiments, an inner surface 142 of the housing 104 may include one or more grooves. For example, the inner surface 142 may include a first groove 144 and/or a second groove 146. In some embodiments, the first groove 144 and/or the second groove 146 may be disposed within the housing 104 between the proximal end 106 and the distal end 108. In some embodiments, the instrument 102 may be disposed within the first groove 144 and/or the second groove 146. In some embodiments, the first groove 144 and/or the second groove 146 may include a support wall 148, another support wall 150 opposite the support wall, and a bottom 152 extending between the support wall 148 and the other support wall 150. In some embodiments, the first groove 144 and/or the second groove 146 may be open opposite the bottom 152. In some embodiments, the first groove 144 and/or the second groove 146 may be linear and/or configured to guide the instrument 102 as the instrument 102 is advanced distally and/or retracted proximally.

In some embodiments, the advancement element 122 may include an arc-shaped channel 154, which may be U-shaped. In some embodiments, the instrument 102 may extend and move through the arc-shaped channel 154. In some embodiments, the first end 128 of the instrument 102 may be fixed. In some embodiments, the first end 128 of the instrument may be fixed within the housing 104. In some embodiments, in response to movement of the advancement element 122 a first distance, the second end of the instrument 102 may be configured to advance distally a second distance. In some embodiments, the second distance may be twice the first distance. In some embodiments, the second distance may be more than twice the first distance. In these and other embodiments, the instrument 102 may extend through multiple U-shapes or other arc-shapes.

In some embodiments, because the first groove 144 and/or the second groove 146 are open opposite the bottom 152, the instrument 102 may tend to buckle in response to the advancement element 122 being advanced distally, as illustrated, for example, in FIG. 1B. Thus, in some embodiments, one or more support features may be used. The support features may be described further in U.S. patent application Ser. No. 17/701,124, filed Mar. 22, 2022, which is herein incorporated by reference in its entirety.

In some embodiments, the distal end 108 of the housing 104 may include a compressible portion 156 proximate a chamber 158 configured to hold blood. In some embodiments, the compressible portion 156 may be constructed of a flexible material, which may have a lower durometer than portions of the housing 104 surrounding the compressible portion 156. In some embodiments, a blood collection pathway 159 may extend within the chamber 158 and/or through the extension tube 132. In some embodiments, in response to compression of the compressible portion 156, the housing 104 may be configured to dispense blood distally from the chamber 608 through the distal end 108 of the housing 104, such as the blunt cannula 127, which may include a distal opening. In some embodiments, the blunt cannula 127 may be configured to limit a volume and/or speed at which blood is dispensed, to ensure that a small sample can be dispensed in a controlled manner.

In some embodiments, in response to compression of the compressible portion 156, the housing 104 may be configured to dispense a small amount of blood for point-of-care testing, which may deliver fast results without a finger prick or other procedure that may be painful for the patient. In some embodiments, a volume of blood dispensed in response to compression of the compressible portion 156 may be suitable for a cartridge for systems such as iSTAT (available from Abbott) or a test strip for systems such as glucose monitors. In some embodiments, the housing 104 may be configured to dispense a single drop of blood or a couple of drops in response to compression of the compressible portion 156.

In some embodiments, the distal end 108 of the housing 104 may include the distal connector 124, and the distal connector 124 may include the compressible portion 156 and the chamber 158. In some embodiments, the compressible portion 156 may include an entirety or a portion of the distal connector 124. In some embodiments, the compressible portion 156 may be proximate the blood collection pathway 159 or the chamber 158. In some embodiments, the compressible portion 156 may surround the blood collection pathway 159 or the chamber 158 disposed within the distal connector 124 and/or the distal end 108 of the housing 104. In some embodiments, the compressible portion 156 may be on opposite sides of the blood collection pathway 159 or the chamber 158 to facilitate pinching and dispensing of blood.

In some embodiments, the compressible portion 156 may include an elastomeric material, which may be molded to form the compressible portion 156. In some embodiments, a durometer and/or thickness of the elastomeric material may vary to achieve a particular force needed to flex the compressible portion 156. In some embodiments, the compressible portion 156 may include an over molded component.

In some embodiments, the compressible portion 156 may include a tab 160, which may form an upper surface of the compressible portion 156. In some embodiments, the tab 160 may include one or more grips or ribs. In some embodiments, the tab 160 may include the elastomeric material. In some embodiments, the distal connector 124 may include the pair of opposing lever arms 126a,126b, and the tab 160 may be disposed between the pair of opposing lever arms 126a,126b, which may facilitate pushing or pinching of the tab 160 by a user, which may result in compression of the compressible portion 156 and dispensing of blood from the housing 104.

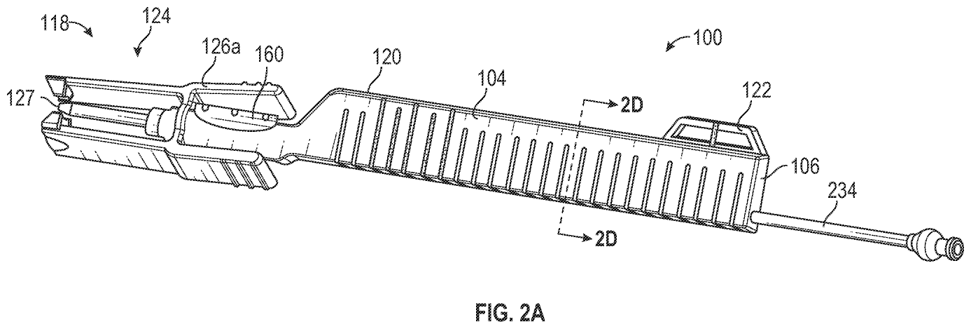

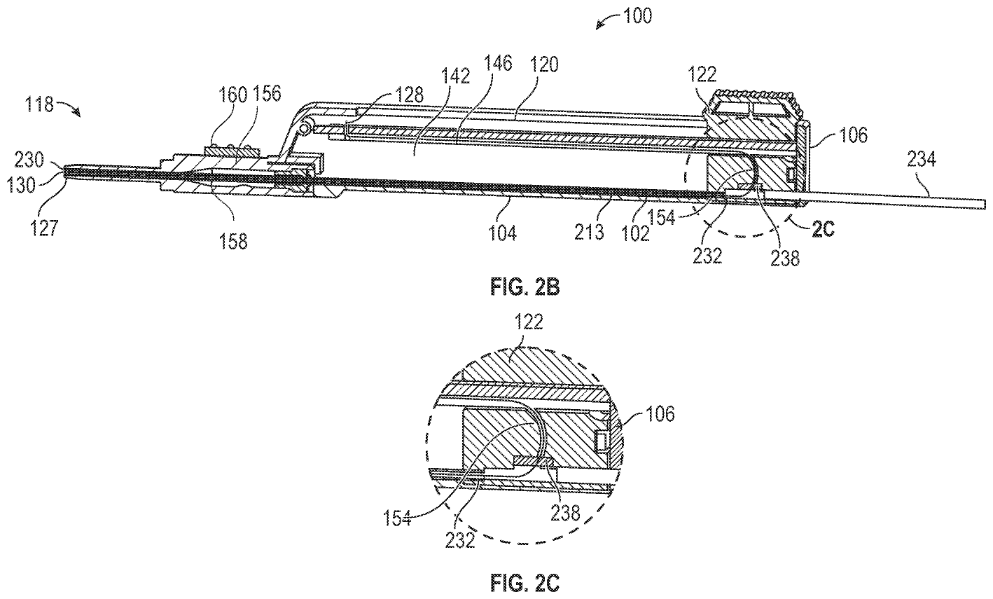

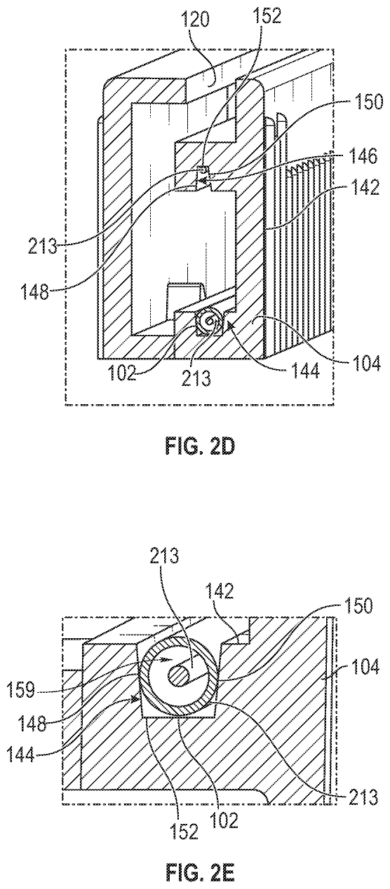

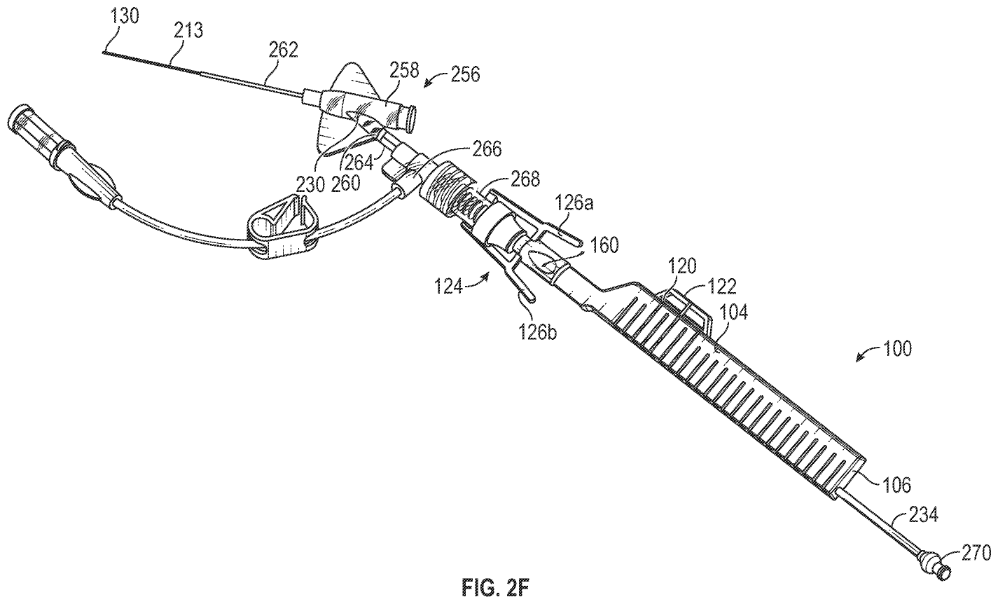

Referring now to FIGS. 2A-2F, in some embodiments, the instrument advancement device 100 may be configured to deliver the instrument 102 and/or another instrument 213 into or through the catheter assembly. In some embodiments, the instrument may include the guidewire, and the other instrument may include a tubing. In some embodiments, the instrument advancement device 100 may provide needle-free delivery of the other instrument 213 and/or the instrument 102 into or through the catheter assembly.

In some embodiments, the other instrument 213 and/or the instrument 102 may be advanced through the catheter of the catheter assembly to push past any occlusions in the catheter or vasculature (e.g., a thrombus or a fibrin sheath at a tip of the catheter, vein collapse, valves, etc.) to create a clear pathway for fluid flow into the catheter assembly, which may aid in blood collection. In some embodiments, the other instrument 213 and/or the instrument 102 may reduce or remove occlusions, improving patency of the catheter for medication and fluid delivery, as well as blood acquisition during a dwell time of the catheter. In some embodiments, the instrument advancement device 100 may improve a blood collection flow rate, blood sample quality, and fluid path robustness, while maintaining a small size to facilitate handling by the user.

In some embodiments, the catheter may include a peripheral intravenous (IV) catheter, a peripherally-inserted central catheter, or a midline catheter. In some embodiments, the catheter through which the other instrument 213 and/or the instrument 102 are delivered may have been previously inserted into vasculature of a patient and may be dwelling within the vasculature when the other instrument 213 and/or the instrument 102 is advanced into the catheter assembly.

In some embodiments, the other instrument 213 and/or the instrument 102 may be disposed within the housing 104, which may be configured to protect the other instrument 213 from damage and/or contamination from a surrounding external environment. In some embodiments, the housing 104 may be rigid or semi-rigid. In some embodiments, the housing 104 may be made of one or more of stainless steel, aluminum, polycarbonate, metal, ceramic, plastic, and another suitable material. In some embodiments, the housing 104 may include the proximal end 106, the distal end 108, and the slot 120. In some embodiments, the slot 120 may extend parallel to a longitudinal axis of the housing 104.

In some embodiments, the instrument advancement device 100 may include an advancement element 122, which may extend through the slot 120 and may be configured to move linearly along the slot 120 between a retracted position illustrated, for example, in FIG. 2A, and an advanced position distal to the retracted position. In some embodiments, the retracted position may correspond to a fully retracted position with the advancement element 122 at a proximal end of the slot 120. In some embodiments, the user may pinch or grasp the advancement element 122 to move the advancement element 122 between the retracted position and the advanced position. In some embodiments, the distal end 108 of the housing 104 may include the distal connector 124. In some embodiments, the distal connector 124 may include opposing lever arms 126a,126b or another suitable connector.

In some embodiments, the instrument 102 may include the first end 128 and the second end 130. In some embodiments, the first end 128 of the instrument 102 may be secured within the housing 104. For example, the first end 128 of the instrument 102 may be fixed to an inner surface of the housing 104. In some embodiments, in response to movement of the advancement element 122 distally a first distance along the slot 120, the second end 130 of the instrument 102 may be configured to advance a second distance. In some embodiments, the second distance may be twice the first distance. In these embodiments, the advancement element 122 and the instrument 102 may have a 1:2 advancement ratio such that for a particular distance the advancement element 122 is moved along the slot 120, the second end 130 of the instrument 102 is moved twice the particular distance.

In some embodiments, the instrument 102 may include any suitable shape. For example, the instrument 102 may include a coil or another suitable fluid permeable structure. In some embodiments, the second end 130 of the instrument 102 may be blunt and/or rounded to prevent damage to vasculature of a patient.

In some embodiments, the other instrument 213 may include a distal end 230 and a proximal end 232. In some embodiments, in response to movement of the advancement element 122 distally the first distance along the slot 120, the distal end 230 of the other instrument 213 may be configured to advance the first distance. In these embodiments, the advancement element 122 and the other instrument 213 may have a 1:1 advancement ratio such that for a particular distance the advancement element 122 is moved along the slot 120, the distal end 230 of the other instrument 213 is moved a distance equal to the particular distance.

In some embodiments, the proximal end 232 of the other instrument 213 may be coupled to the advancement element 122. In some embodiments, the instrument advancement device 100 may include an extension tube 234 coupled to the advancement element 122, and a blood collection pathway 159 may extend through the other instrument 213, the advancement element 122, and the extension tube 234.

In some embodiments, the instrument advancement device 100 may include a septum or seal 238 disposed within the advancement element 122 and preventing fluid communication between the blood collection pathway 159 and a portion of a guidewire pathway through which the instrument 102 moves. In some embodiments, the seal 238 may include an elastomeric septum. In some embodiments, the instrument 102 may include the coil or other fluid permeable structure, which may be distal to the seal 238 or extend through the seal 238 when the advancement element 122 is in an initial or fully retracted position. In some embodiments, the instrument 102 may be disposed proximal to the compressible portion 156 and/or the chamber 158 such that compression of the compressible portion 156 does not pinch the instrument 102.

In some embodiments, in response to movement of the advancement element 122 distally the first distance along the slot 120, the second end 130 of the instrument 102 and the distal end 230 of the other instrument 213 may move from inside the housing 104 to outside of the housing 104. In some embodiments, in response to the advancement element 122 being disposed at a proximal end of the slot 120, the second end 130 of the instrument 102 and the distal end 230 of the other instrument 213 may be aligned with or proximal to a distal end 40 of the blunt cannula or male luer of the distal connector 124, which may protect the instrument 102 and the other instrument 213 and prevent contamination thereof.

In some embodiments, the inner surface 142 of the housing 104 may include one or more grooves, which may be disposed between the proximal end 106 of the housing 104 and the distal end 108 of the housing 104. For example, the inner surface 142 may include the first groove 144 and/or the second groove 146. In some embodiments, the first groove 144 and/or the second groove 146 may be disposed within the housing 104 between the proximal end 106 and the distal end 108. In some embodiments, the other instrument 213 may be disposed within the first groove 144, which may provide guidance of the other instrument 213.

In some embodiments, the instrument 102 may be disposed within the first groove 144 and the second groove 146, which may provide guidance for the instrument 102. In some embodiments, the first groove 144 and/or the second groove 146 may include the support wall 148, another support wall 150 opposite the support wall, and the bottom 152 extending between the support wall 148 and the other support wall 150. In some embodiments, the first groove 144 and/or the second groove 146 may be open opposite the bottom 152. In some embodiments, the first groove 144 and/or the second groove 146 may be linear and/or configured to guide the instrument 102 as the instrument 102 is advanced distally and/or retracted proximally.

In some embodiments, the instrument 102 may be disposed in the first groove 144 and/or the second groove. In some embodiments, the other instrument 213 may be disposed within the first groove 144. In some embodiments, the first groove 144 and/or the second groove 146 may extend from the distal end 108 towards the proximal end 106 along all or a portion of a path on which the advancement element 122 travels. In some embodiments, the instrument advancement device 100 may include a support feature, which may be configured to contact the other instrument 213 to prevent the other instrument 213 and/or the instrument 102 from buckling. Some example support features are further described in U.S. patent application Ser. No. 17/701,124, filed Mar. 22, 2022. In some embodiments, the advancement element 122 may include an arc-shaped channel 154, which may be U-shaped. In some embodiments, the instrument 102 may extend and move through the arc-shaped channel 154. In some embodiments, in response to movement of the advancement element 122 a first distance, the second end of the instrument 102 may be configured to advance distally a second distance that is more than twice the first distance. In these and other embodiments, the instrument 102 may extend through multiple arc-shapes.

In some embodiments, the compressible portion 156 may function in a similar fashion as illustrated in FIG. 1C. In some embodiments, the other instrument 213, which may include the blood collection pathway 159, may be disposed within the chamber 158. In some embodiments, compression, pushing, or squeezing of the compressible portion 156 by the user may pinch the other instrument 213, which may expel blood distally from the other instrument 213 through the distal end 118 or the blunt cannula 127 into a point-of-care device.

Referring now to FIG. 2F, the advancement element 122, the other instrument 213, and the instrument 102 are illustrated in the advanced position, according to some embodiments. In some embodiments, the advancement element 122 may be disposed at a distal end of the slot 120 when the advancement element 122 is in the advanced position. In some embodiments, the distal end 230 of the other instrument 213 may be disposed within a portion of a catheter assembly 256 when the advancement element 122 is in the advanced position.

In some embodiments, the catheter assembly 256 may include a catheter adapter 258, which may include a distal end, a proximal end, and a fluid pathway extending therethrough. In some embodiments, the catheter adapter 258 may include a side port 260 in fluid communication with the fluid pathway of the catheter adapter 258. In some embodiments, the catheter assembly 256 may include a catheter 262, which may be secured within the catheter adapter 258 and may extend distally from the distal end of the catheter adapter 258. In some embodiments, the catheter assembly 256 may be inserted into vasculature of a patient via an introducer needle (not illustrated), which may extend through the catheter 262 and may be used to pierce skin and vasculature of the patient to insert the catheter 262. In some embodiments, the introducer needle may be removed from the catheter assembly 256 after the catheter 262 is placed in the vasculature.

In some embodiments, an extension tube 264 may extend proximally from the side port 260 and/or may be connected to a T-connector 266 or another suitable connector. In some embodiments, the T-connector 266 or the other suitable connector may be connected to a needleless connector 268, which may be connected to the distal connector 124 of the instrument advancement device 100.

In some embodiments, in response to the catheter 262 being inserted into the vasculature, blood may be configured to flow proximally from the catheter 262 through one or more of the catheter adapter 258, the side port 260, the extension tube 264, the T-connector 266, and the needleless connector 268. In some embodiments, the blood may then be configured to flow into the distal connector 124 and proximally through the blood collection pathway 159 of the instrument advancement device 100. In some embodiments, a proximal end of the extension tube 234 may be integrated with a connector 270 which may be coupled to or monolithically formed with a blood collection device, such as, for example, a syringe, a BD VACUTAINER® blood collection tube available from Becton Dickinson & Company of Franklin Lakes, N.J., or another suitable blood collection device. In these and other embodiments, the blood collection device may include a vacuum tube receiver having a needle covered by a protective sheath.

As illustrated in FIG. 2F, in some embodiments, when the advancement element 122 is in the advanced position, the distal end 230 of the other instrument 213 may be disposed within the side port 260 or elsewhere within the catheter adapter 258. As mentioned, in some embodiments, the other instrument 213 may extend through the catheter 262 when the advancement element 122 is in the advanced position. In some embodiments, a maximum outer diameter of the other instrument 213 may be less than portions of the catheter assembly 256 through which the other instrument 213 is configured to extend. In some embodiments, the maximum outer diameter of the other instrument 213 may be increased to a maximum size that will still fit through the portions of the catheter assembly 256 through which the other instrument 213 is configured to extend. In some embodiments, a maximum outer diameter of the instrument 102, which may include the coil, may be less than a minimum inner diameter of the other instrument 213 to facilitate movement of the instrument 102 through the other instrument 213.

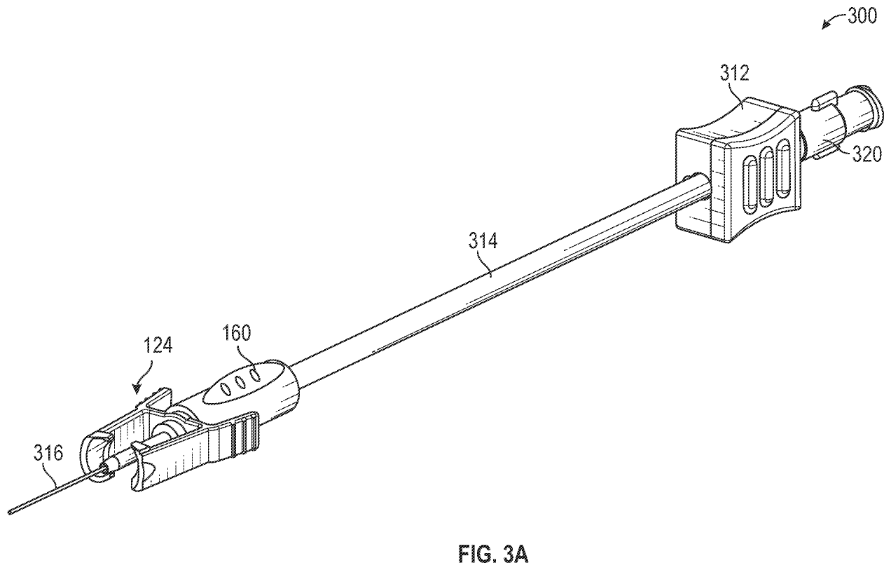

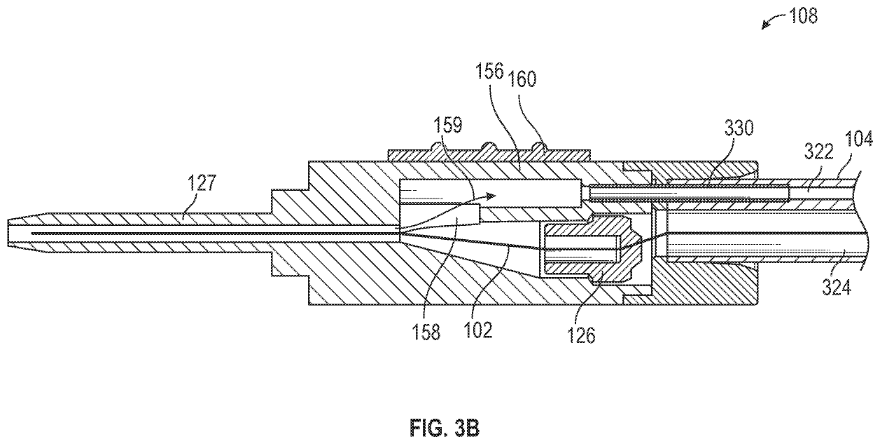

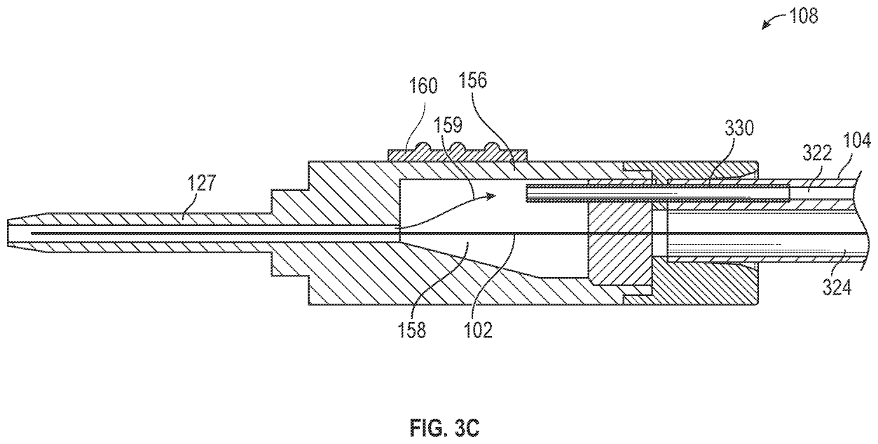

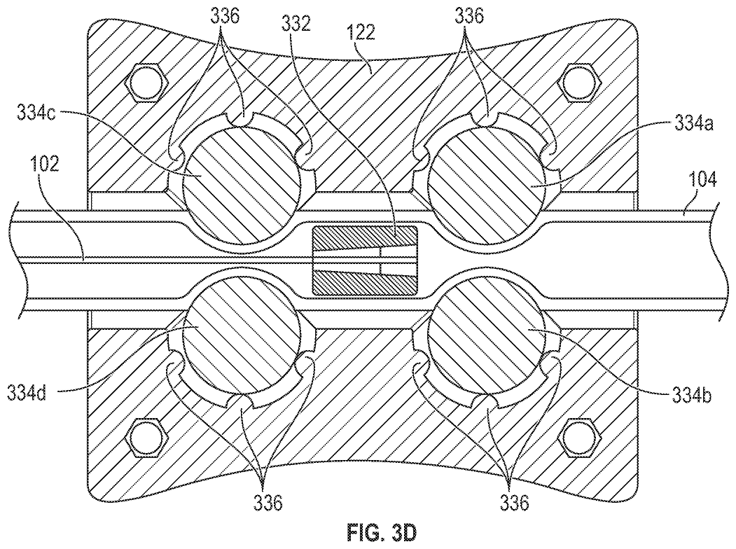

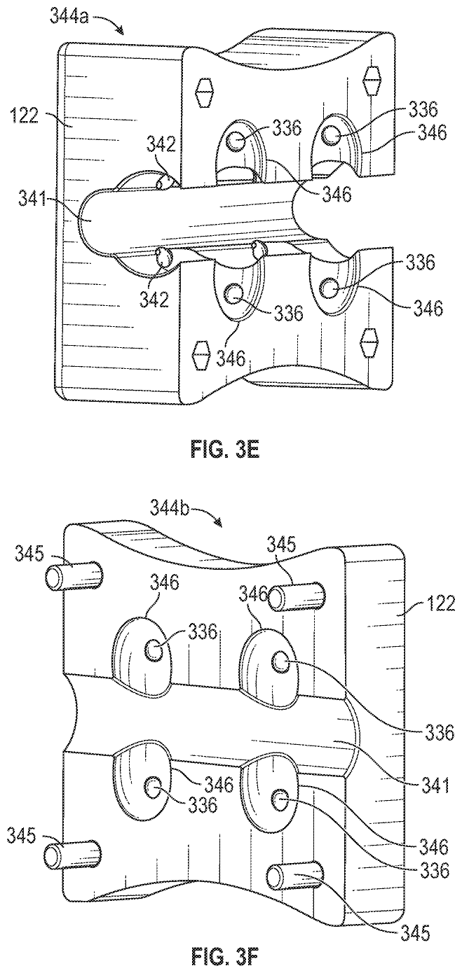

Referring now to FIGS. 3A-3F, an instrument advancement device 300 is illustrated, according to some embodiments. In some embodiments, the instrument advancement device 300 may be similar or identical to the instrument advancement device 100 of FIGS. 1-2 in terms of one or more components and/or operation. In some embodiments, the instrument advancement device 300 may include an advancement element 312 and a housing 314 extending through the advancement element 312. In some embodiments, the housing 314 may include an extension tube, which may be flexible or semi-flexible. In some embodiments, the housing 314 may include a coextruded guidewire to add stiffness to the housing 314.

In some embodiments, the instrument advancement device 300 may include the instrument 102, which may include the guidewire or another suitable instrument. In some embodiments, the instrument 102 may be colored to increase visibility. In some embodiments, the guidewire may be constructed of metal or another suitable material. In these and other embodiments, the instrument 102 may be lubricated or coated to ease advancement. In some embodiments, the tube may be soft or stiff. In some embodiments, the housing 314 may create a closed path for blood flow and/or reduce contamination of the blood due to drug adsorption in a catheter assembly.

In some embodiments, the instrument 102 may include a guidewire, which may include a spring or coil. In some embodiments, the spring or coil may include varying pitches along a length of the spring or coil. For example, a pitch of the spring or coil upstream from or proximal to a catheter tip may be larger to facilitate more blood flow and increase flow rate, and a pitch of the spring or coil near the catheter tip may be smaller to prevent blood clots from entering the catheter tip, while still allowing blood to flow through it. In some embodiments, the guidewire may include a rod, which may extend through a center portion of the spring or coil. In some embodiments, the guidewire may include the rod and may not include the spring or coil.

Over time a catheter can become occluded at the catheter tip due to presence of fibrin sheath, thrombus, or vein walls or valves. In some embodiments, the instrument 102 may be configured to extend into and/or through the catheter assembly to push through and/or disrupt an occlusion of the catheter. In some embodiments, the instrument 102 may overcome thrombus and fibrin sheath in or around the catheter assembly or in the vein that might otherwise prevent blood draw. In some embodiments, the instrument advancement device 300 may reduce trauma to the vasculature while also facilitating fluid delivery, blood collection, patient or device monitoring, or other clinical needs. In some embodiments, the instrument advancement device 300 may decrease hemolysis and reduce blood exposure. In some embodiments, the instrument 102 may include a vascular access instrument configured to advance distally through the catheter assembly and into the vasculature of the patient.

In some embodiments, a distal end of the instrument advancement device 300 may include the distal connector 124 or another suitable connector. In some embodiments, the distal connector 124 may be configured to couple to the catheter assembly, which may be existing or already dwelling within the vasculature of the patient. In some embodiments, the catheter assembly may include a catheter adapter, which may include a distal end, a proximal end, and a lumen extending through the distal end of the catheter adapter and the proximal end of the catheter adapter. In some embodiments, the catheter may extend from the distal end of the catheter adapter. In some embodiments, the catheter may include a peripheral intravenous catheter, a midline catheter, or a peripherally inserted central catheter. In some embodiments, the catheter assembly may include an introducer needle, which may extend through the catheter and facilitate piercing of skin and the vasculature to insert the catheter into the patient. In some embodiments, the introducer needle may be removed from the catheter assembly prior to coupling of the instrument advancement device 300 to the catheter assembly.

In some embodiments, the catheter assembly may be straight. In other embodiments, the catheter assembly may be integrated, having an extension tube that is integrated with the catheter adapter. In some embodiments, the catheter assembly may include an extension set, which may include the extension tube extending from and integrated with a side port of the catheter adapter. In some embodiments, the distal connector 124 may be configured to couple to a portion of the catheter assembly, such as the proximal end of the catheter adapter and/or a needleless access connector. In some embodiments, the needleless access connector may be coupled to a proximal end, a T-connector, or another portion of the extension set. In some embodiments, the needleless access connector may be permanently connected, such as, for example, via adhesive, to the distal connector 124 to prevent intentional or unintentional removal by a user.

In some embodiments, a distal end of the housing 314 may be coupled to the distal connector 124. In some embodiments, a proximal end of the housing 314 may be coupled to a proximal adapter 320, which may include another distal adapter or another suitable connector. In some embodiments, the proximal adapter 320 may be configured to couple to a blood collection device. In some embodiments, the blood collection device may include a syringe, a BD VACUTAINER® one-use holder (available from Becton, Dickinson and Company of Franklin Lakes, N.J.), a BD VACUTAINER® LUER-LOK™ access device (also available from Becton, Dickinson and Company of Franklin Lakes, New Jersey), or another suitable blood collection device, which may provide suction.

In some embodiments, the instrument 102 may be advanced prior to or during infusion or blood draw. In some embodiments, after completing a blood draw or infusion and before uncoupling the instrument advancement device 300 from the catheter assembly, the user may retract the instrument by moving the advancement element 312 backward or proximally. Thus, in some embodiments, a risk of exposure of the user to blood may be decreased.

Referring now to FIG. 3B, in some embodiments, the housing 314 may include a first lumen 322 and a second lumen 324, which may be separate from the first lumen 322 along an entire length of the housing 314. In some embodiments, a blood collection pathway may extend through the first lumen 322. In some embodiments, the instrument 102 may be disposed within the second lumen 324. In some embodiments, a diameter of the second lumen 324 may be larger than a diameter of the first lumen 322. In some embodiments, the diameter and/or a length of the first lumen 322 may be selected based on a desired flow rate and/or to reduce hemolysis.

In some embodiments, in response to moving the advancement element 312 distally along the housing 314, the instrument 102 may be advanced distally within the second lumen 324. In some embodiments, in response to moving the advancement element 312 proximally along the housing 314, the instrument 102 may be retracted proximally within the second lumen 324.

In some embodiments, the instrument advancement device 300 may include the septum 136 disposed within the distal connector 124 and configured to seal the second lumen 324 or prevent blood flow into the second lumen 324. In these and other embodiments, the septum 136 may not seal the first lumen 322 such that blood may flow proximally along a blood collection pathway from the distal connector 124 through the first lumen 322 for blood collection. In some embodiments, the septum 136 may be elastomeric.

In some embodiments, a distal end of the instrument 102 may be disposed proximal to a distal end of the distal connector 124 when the advancement element 312 is fully retracted in a proximal direction. In some embodiments, the distal end of the instrument 102 may be disposed proximal to the septum 136 when the advancement element 312 is fully retracted in the proximal direction and/or the instrument 102 may be sealed within the housing 314.

In some embodiments, the instrument advancement device 300 may include a cannula 330, which may connect a distal end of the first lumen 322 and the distal connector 124. In some embodiments, the cannula 330 may be blunt. In some embodiments, the blood collection pathway 159 may extend through the cannula 330, which may prevent blood leakage. In some embodiments, the cannula 330 may be constructed of steel, plastic, metal, or another suitable material. In some embodiments, the cannula 330 may be coupled to the distal connector 124 or monolithically formed with the distal connector 124 as a single unit. In some embodiments, the septum 136 may be concentric with the second lumen 324 or offset slightly to obtain adequate wall thicknesses.

Referring now to FIG. 3C, in some embodiments, the septum 136 may extend across a width of an inner lumen of the distal connector 124. In some embodiments, the septum 136 may seal the second lumen 324 or prevent blood flow into the second lumen 324. In some embodiments, the cannula 330 may extend from the first lumen 322 through the septum 136 to allow fluid flow therethrough.

Referring now to FIG. 3D, in some embodiments, the instrument advancement device 300 may include a wedge 332 disposed within the advancement element 312 and the second lumen 324 of the housing 314. In some embodiments, the instrument advancement device 300 may include a pair of opposing pinch members 334 configured to pinch the housing 314. In some embodiments, the pair of opposing pinch members 334a,b may be disposed within the advancement element 312 proximal to the wedge 332 and configured to move along the housing 314 with the advancement element 312. In some embodiments, the housing 314 may include a multi-lumen extension tube. In other embodiments, the housing 314 may include a single lumen extension tube, and blood may be configured to flow through the single lumen extension tube through and/or around the wedge 332.

In some embodiments, the instrument 102 may extend distally from the wedge 332. In some embodiments, the instrument 102 may be disposed within the second lumen 324. In some embodiments, in response to moving the advancement element 312 distally along the housing 314, the pair of opposing pinch members 334a,b may push the wedge 332 distally, and the instrument 102 may be advanced distally.

In some embodiments, the instrument advancement device 300 may include another pair of opposing pinch members 334c,d configured to pinch the housing 314. In some embodiments, the other pair of opposing pinch members 334c,d may be disposed within the housing distal to the wedge 332 and configured to move along the housing 314 with the advancement element 312. In some embodiments, in response to moving the advancement element 312 proximally along the housing 314, the pair of opposing pinch members 334c,d may push the wedge 332 proximally and the instrument 102 may be retracted proximally.

The pair of opposing pinch members 334a,b and the other pair of opposing pinch members 334c,d may be referred to collectively in the present disclosure as “opposing pinch members 334.” In some embodiments, in response to movement of the advancement element 312 along the housing 314, the opposing pinch members 334 may rotate with respect to the advancement element 312 and the housing 314. In some embodiments, in response to movement of the advancement element 312 along the housing 314, the opposing pinch members 334 may rotate with respect to the advancement element 312 and the housing 314, which may rotate the instrument 102. In some embodiments, an inner surface of the advancement element 312 may include one or more bumps 336 in contact with the opposing pinch members 334, which may reduce friction as the opposing pinch members 334 rotate. In some embodiments, the wedge 332 and/or the opposing pinch members 334 may be lubricated with a lubricant, which may reduce friction.

In some embodiments, the opposing pinch members 334 may be constructed of plastic, metal, or another suitable material. In some embodiments, the opposing pinch members 334 may include spherical balls, ball bearings, wheels, or cylinders, which may be configured to rotate with respect to the advancement element 312. In some embodiments, the opposing pinch members 334 may include the wheels, which may be smooth or include feet along their edges. In these embodiments, lubricant may be applied to axels of the wheels to reduce friction. In some embodiments, the opposing pinch members 334 may be fixed with respect to the advancement element 312. For example, the opposing pinch members 334 may be molded into the advancement element 312.

In some embodiments, a number of the opposing pinch members 334 may vary based on a shape of the wedge 332. In some embodiments, the instrument advancement device 300 may include the pair of opposing pinch members 334a,b and the other pair of opposing pinch members 334c,d in response to the shape of the wedge 332 being cylindrical, for example. In some embodiments, the instrument advancement device 300 may include a single pair of the opposing pinch members 334, such as the pair of the opposing pinch members 334a,b, in response to the wedge 332 including a dog bone shape, and the single pair may be disposed in a middle or depression of the dog bone shape.

Referring now to FIG. 3E-3F, in some embodiments, the advancement element 312 may include an aperture 341 extending therethrough and configured to receive the housing 314. In some embodiments, the inner surface of the advancement element 312 may include one or more protrusions 342, which may contact the housing 314 to reduce friction between the housing 314 and the advancement element 312 as the advancement element 312 moves along the housing 314. In some embodiments, the advancement element 312 may include multiple pieces 344a,b, which may be coupled together via pegs 345 or any other suitable mechanism. In other embodiments, the advancement element 312 may be monolithically formed as a single unit.

In some embodiments, the advancement element 312 may include multiple cutouts 346, which may include the bumps 336. In these and other embodiments, the opposing pinch members 334 may include the spherical balls. In some embodiments, the cutouts 346 may be generally spherical and/or may extend outwardly from the aperture 341. In some embodiments, halves of the cutouts 346 illustrated in the multiple pieces 344a,b may be joined together to form the cutouts 346.

In some embodiments, the advancement element 312 may be rigid or semi-rigid to facilitate gripping and/or one-handed advancement by the user. In some embodiments, the advancement element 312 may include one or more grip features or a shape to facilitate gripping by the user. In some embodiments, the grip features may include one or more of ridges, indents, and tabs on a top of the advancement element 312 and/or one or more sides of the advancement element 312. In some embodiments, the shape of the advancement element 312 may include a square, cylinder, dog bone, or another suitable shape. In some embodiments, the shape of the advancement element 312 and/or the grip features may facilitate the user advancing and/or retracting the instrument 102 without contacting the instrument, thereby decreasing a risk of contamination and/or infection. In some embodiments, the advancement element 312 may include one or more textured surfaces to facilitate gripping by the user.

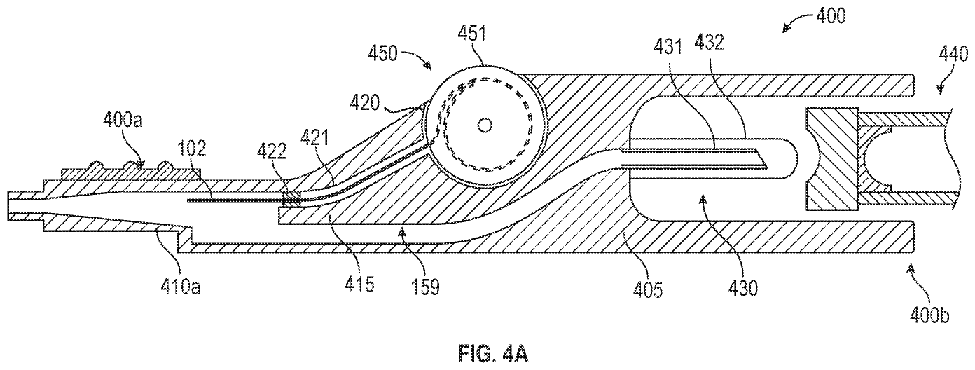

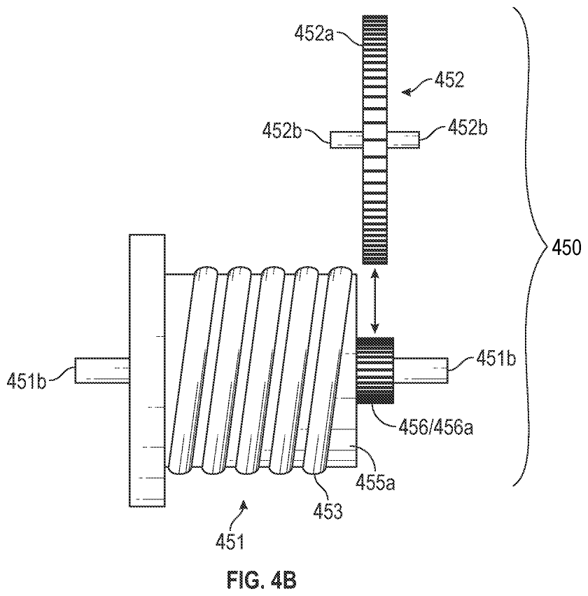

Referring now to FIGS. 4A-4B, an instrument advancement device 400 is illustrated, according to some embodiments. In some embodiments, the instrument advancement device 400 may be similar or identical to the instrument advancement device 100 of FIGS. 1-2 and/or the instrument advancement device 300 of FIG. 3 in terms of one or more features and/or operation. In some embodiments, the instrument advancement device 400 may include a housing 405 having a distal end 400a, which may be configured in any manner described above, and a proximal end 400b that forms a vacuum tube receiver 430 having a needle 431 covered by a protective sheath 432. In some embodiments, the blood collection pathway 159 may extend within the instrument advancement device 400 from the needle 431 to the distal end 400a. Accordingly, when a vacuum tube 440 is inserted into the vacuum tube receiver 430, a blood sample can be collected through the blood collection pathway 159. In other embodiments, the proximal end 400b may include a Luer connector or any other type of connector that is coupled to the blood collection pathway 159.

In some embodiments, the instrument advancement device 400 may include an advancement element 450 that enables an instrument 102 to be advanced in a distal direction through an IV catheter and/or subsequently withdrawn in a proximal direction. In some embodiments, a compartment 420 may be formed within the instrument advancement device 400 and may houses the advancement element 450. In some embodiments, a dividing wall 415 may create an instrument channel 421 that extends distally from the compartment 420 and joins the blood collection pathway 159 at a distal end 410a of the blood collection pathway 159. In some embodiments, the distal end 410a may include the distal connector 124. In some embodiments, a seal 422 may be positioned within and the span the instrument channel 421 to isolate the instrument channel 421 from the blood collection pathway 159.

As illustrated in FIG. 4B, which is an isolated rear view of the advancement element 450, the advancement element 450 may include a spool 451 having an axle 451b that maintains spool 451 within compartment 420 and allows spool 451 to rotate. In some embodiments, the spool 451 may include a spool drum 451c around which the instrument 102 is wound. In some embodiments, a portion of the spool 451 may form an advancement wheel 251a that extends upwardly from the compartment 420. Accordingly, a user can directly rotate the spool 451 by applying a force to the advancement element 450. Such rotation can cause the instrument 102 to be advanced and retracted within the instrument channel 421 depending on the direction of rotation. FIG. 4 is just one example of an instrument delivery device that includes an advancement wheel and the compressible portion 156. It is understood that, in some embodiments, other instrument advancement devices not described or illustrated in the present disclosure may include the compressible portion 156.

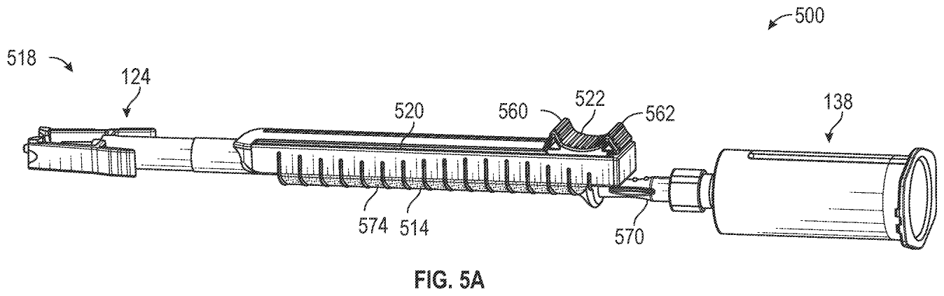

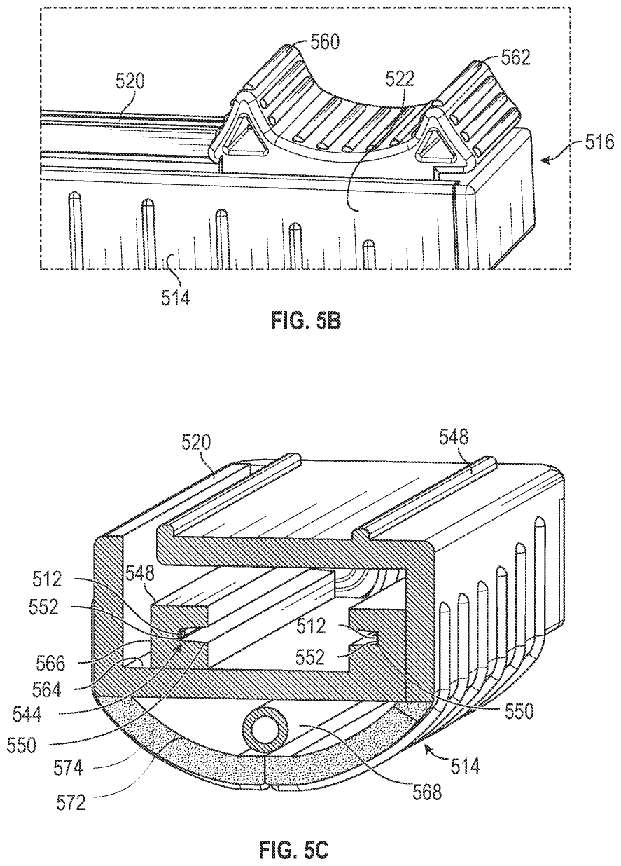

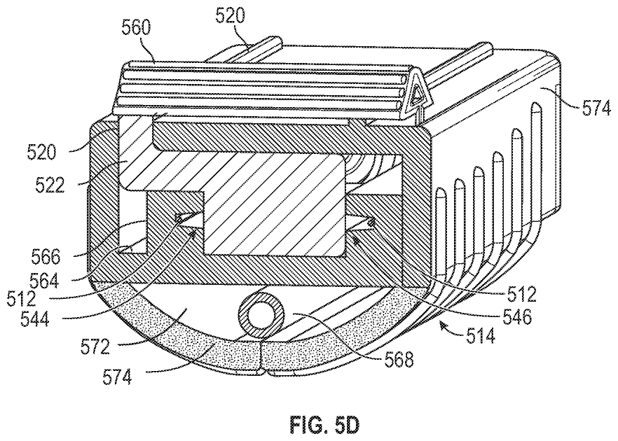

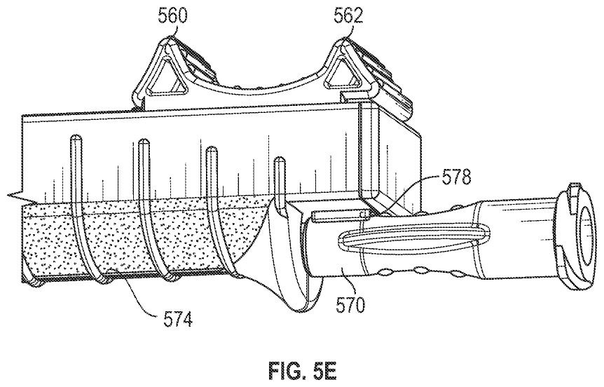

Referring now to FIGS. 5A-5E, the instrument advancement device 500 is illustrated, according to some embodiments. In some embodiments, the instrument advancement device 500 may be similar or identical to one or more of the following: the instrument advancement device 100 of FIGS. 1-2, the instrument advancement device 300 of FIG. 3, and the instrument advancement device 400 of FIG. 4 in terms of one or more features and/or operation.

In some embodiments, an upper surface of the advancement element 522 may include a first push tab 560 and a second push tab 562, which may allow the user to advance the instrument 102 in a distal direction without repositioning his or her hand. The advancement element 522 may otherwise need to be slid further in the distal direction than an average hand size can slide the advancement element 522 in one push, and the user would reposition his or her hand to continue to push the advancement element 522 in the distal direction. In some embodiments, to advance the instrument 102 from the retracted position to the advanced position, the user may reposition his or her finger but not his or her hand grip due to the second push tab 562 in addition to the first push tab 560. In some embodiments, the advancement element 522 may include more than two push tabs, which may extend a length of the advancement element 522.

In some embodiments, the first push tab 560 may be distal to the second push tab 562 and/or at a distal end of the advancement element 522. In some embodiments, the second push tab 562 may be at a proximal end of the advancement element 522. In some embodiments, the first push tab 560 and the second push tab 562 may be a same height, which may facilitate securement of the finger of user. In some embodiments, the first push tab 560 and the second push tab 562 may be different heights. For example, the first push tab 560 may be taller than the second push tab 562, which may make the upper surface of the advancement element 522 easier to push on if the finger cannot fit between the first push tab 560 and the second push tab 562.

In some embodiments, the housing 514 is rotated from FIGS. 5A-5E such that the instrument 102 is disposed in a horizontal plane instead of a vertical plane. In these and other embodiments, the slot 520 may be disposed on a top of the housing 514. In some embodiments, positioning of the instrument 102 in the horizontal plane may change an aspect ratio of the instrument advancement device 500 from tall and narrow to short and wide, which may facilitate the instrument advancement device 500 lying flat in a hand of the user and may make the instrument advancement device 500 easier to grip.

In some embodiments, the housing 514 may include a dead well 564 aligned with the slot 520 and separated from the first groove 544 and/or the second groove 546 by a joiner wall 566 that may form the bottom 552 and join the support wall 548 and another support wall 550. In some embodiments, the dead well 564 may include a pocket where any contaminants coming through the slot 520 may get caught. In some embodiments, the dead well 564 may be part of a tortuous path that keeps the instrument 102 sterile after a package of the probe advancement device 500 has been opened. In some embodiments, any contaminants coming through the slot 520 must take multiple turns in the tortuous path to reach the instrument 102 disposed within the first groove 544 and the second groove 546, decreasing a likelihood that the contaminants will reach the instrument 102.

In some embodiments, an extension tube 568 may extend from the distal connector 124 and may bypass the fluid seal 536, extending through the housing 514 to a proximal connector 570, which may be disposed at the proximal end 516 of the housing 514 or proximal to the proximal end 516 of the housing 514. In some embodiments, the blood collection device 138 may be coupled to the proximal connector 570. In some embodiments, the blood collection device 138 may include a syringe, a BD VACUTAINER® one-use holder (available from Becton, Dickinson and Company of Franklin Lakes, N.J.), a BD VACUTAINER® LUER-LOK™ access device (also available from Becton, Dickinson and Company of Franklin Lakes, N.J.), or another suitable blood collection device, which may provide suction.

In some embodiments, the extension tube 568 may be disposed in a tunnel or lumen 572 of the housing 514 to reduce a likelihood that the extension tube 568 interferes with movement of the instrument 102. In some embodiments, the extension tube 568 may be used for blood collection and/or fluid infusion. In some embodiments, the extension tube 568 may be constructed of a flexible material.

In some embodiments, a compressible portion 574 of the housing 514 may be proximate the lumen 572. In some embodiments, the extension tube 568 may extend through the lumen 572 and/or may be coupled to the proximal connector 570. In some embodiments, the blood may be configured to flow through the extension tube 568 during blood collection. In some embodiments, in response to compression of the compressible portion 574, the extension tube 568 may be pinched, and the housing 514 may be configured to dispense blood from the extension tube 568 distally through a distal end 518 of the housing 514. In some embodiments, the compressible portion 574 may be aligned with the slot 520 along all or a portion of the slot 520. In FIG. 5A and 5C-5E, the compressible portion 574 includes dotted shading for illustrative purposes.

In some embodiments, the blood collection pathway 159 may extend through the extension tube 568. In some embodiments, blood may be dispensed distally through the blunt cannula 127 of the distal connector 124, which may include a distal opening. In some embodiments, the blunt cannula 127 may be configured to limit a volume and/or speed at which blood is dispensed, to ensure that a small sample can be dispensed in a controlled manner.

In some embodiments, in response to compression of the compressible portion 574, the housing 514 may be configured to dispense a small amount of blood for point-of-care testing, which may deliver fast results without a finger prick or other procedure that may be painful for the patient. In some embodiments, a volume of blood dispensed in response to compression of the compressible portion 574 may be suitable for a cartridge for systems such as iSTAT (available from Abbott) or a test strip for systems such as glucose monitors. In some embodiments, the housing 514 may be configured to dispense a single drop of blood or a couple of drops in response to compression of the compressible portion 574.

In some embodiments, the compressible portion 574 may be constructed of a flexible material, which may have a lower durometer than another portion of the housing 514, such as, for example, an upper body of the housing 514, which may be coupled to the compressible portion 574 and may include the dead well 564, the first groove 544, the second groove 546, and the joiner wall 566. In some embodiments, the compressible portion 574 may include an elastomeric material, which may be molded to form the compressible portion 574. In some embodiments, a durometer and/or thickness of the elastomeric material may vary to achieve a particular force needed to flex the compressible portion 574. In some embodiments, the compressible portion 574 may include an over molded component.

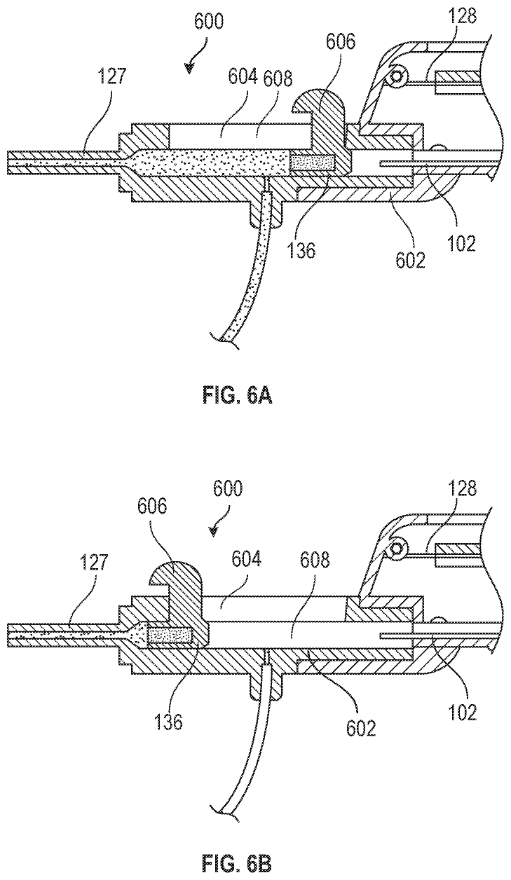

Referring now to FIGS. 6A-6B, in some embodiments, a distal end 600 of a housing 602 may include a slot 604 and a slider 606 configured to move along the slot 604. In some embodiments, in response to movement of the slider 606 from a proximal end of the slot 604 to a distal end of the slot 604, blood may be dispensed distally through the distal end 600 of the housing 602.

In some embodiments, the distal end 600 may be configured to dispense a small amount of blood for point-of-care testing, which may deliver fast results without a finger prick or other procedure that may be painful for the patient. In some embodiments, a volume of blood dispensed may be suitable for a cartridge for systems such as iSTAT (available from Abbott) or a test strip for systems such as glucose monitors. In some embodiments, the distal end 600 may be configured to dispense a single drop of blood or a couple of drops of blood.

In some embodiments, the slider 606 may be coupled to the septum 136 disposed within a chamber 608 of the distal end 600 of the housing 602. In some embodiments, the septum 136 may be configured to move with the slider 606. In some embodiments, the distal end 600 of the housing 602 may include the chamber 608 configured to hold blood, and the septum 136 may extend across the chamber 608, which may facilitate removal of blood within the chamber 608. In some embodiments, the instrument 102 may include the guidewire.

In some embodiments, the distal end 600 of the housing 602 may include the distal connector 124, and the distal connector 124 may include the slider 606, the slot 604, and the septum 126. In some embodiments, the distal connector 124 may include the pair of opposing lever arms 126a,126b. In some embodiments, the slider 606 may be disposed between the pair of opposing lever arms 126a,126b, which may facilitate access to the slider 606 by the user.

In accordance with some embodiments, further details regarding the instrument advancement device 100, the instrument advancement device 300, the instrument advancement device 400, and the instrument advancement device 500 may be described in U.S. patent application Ser. No. 17/574,127, filed Jan. 12, 2022, and U.S. patent application Ser. No. 17/709,935, filed Mar. 31, 2022, which are each hereby incorporated by reference in their entirety.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. An instrument advancement device, comprising:
a housing, comprising a distal end and a proximal end;
an instrument disposed within the housing;
an advancement element, wherein in response to movement of the advancement element with respect to the housing, the instrument is configured to advance distal to the distal end of the housing,
wherein the distal end of the housing comprises a compressible portion proximate a chamber configured to hold blood, wherein in response to compression of the compressible portion, the housing is configured to dispense blood distally from the chamber through the distal end of the housing.

2. The instrument advancement device of claim 1, wherein the instrument comprises a guidewire.

3. The instrument advancement device of claim 2, further comprising another instrument coupled to the advancement element, wherein the other instrument comprises a tubing, wherein blood is configured to flow proximally through the tube, wherein the guidewire is disposed within the tubing.

4. The instrument advancement device of claim 1, wherein the distal end of the housing comprises a distal connector, wherein the distal connector comprises the compressible portion and the chamber.

5. The instrument advancement device of claim 4, wherein the compressible portion further comprising a tab forming an upper surface of the compressible portion.

6. The instrument advancement device of claim 5, wherein the distal connector comprises a pair of opposing lever arms, wherein the tab is disposed between the pair of opposing lever arms.

7. The instrument advancement device of claim 1, wherein the housing comprises a slot, wherein the advancement element extends through the slot and is configured to move linearly along the slot between a retracted position and an advanced position, wherein in response to movement of the advancement element from the retracted position to the advanced position, the instrument is configured to advance distal to the distal end of the housing.

8. The instrument advancement device of claim 1, wherein the housing comprises a slot, wherein the advancement element extends through the slot and is configured to move linearly along the slot, wherein the instrument comprises a guidewire having a first end and a second end, wherein in response to movement of the advancement element distally a first distance along the slot, the second end of the guidewire is configured to advance a second distance, wherein the second distance is at least twice the first distance, further comprising another instrument, wherein the other instrument comprises a tubing comprising a distal end and a proximal end, wherein in response to movement of the advancement element distally the first distance along the slot, the proximal end of the tubing is configured to advance the first distance.

9. The instrument advancement device of claim 1, wherein the housing comprises an extension tube extending through the advancement element, further comprising a wedge disposed within the advancement element and the extension tube, further comprising a pair of opposing pinch members configured to pinch the extension tube, wherein the pair of opposing pinch members are disposed within the advancement element and configured to move along the extension tube with the advancement element, wherein the instrument extends distally from the wedge, wherein in response to moving the advancement element distally along the extension tube, the pair of opposing pinch members push the wedge distally and the instrument is configured to advance distal to the distal end of the housing.

10. An instrument advancement device, comprising:
a housing, comprising a proximal end, a distal end, and a slot;
an advancement element extending through the slot and configured to move linearly along the slot between a retracted position and an advanced position; and
an instrument comprising a first end and a second end, wherein in response to movement of the advancement element linearly along the slot from the retracted position to the advanced position, the instrument is advanced beyond the distal end of the housing,
wherein the housing comprises a compressible portion, wherein in response to compression of the compressible portion, the housing is configured to dispense blood distally through the distal end of the housing.

11. The instrument advancement device of claim 10, wherein the instrument comprises a guidewire.

12. The instrument advancement device of claim 10, wherein the housing comprises a lumen configured for blood to flow therethrough, wherein the compressible portion is proximate the lumen.

13. The instrument advancement device of claim 12, wherein the proximal end of the housing comprises a proximal connector, further comprising an extension tube extending through the lumen and coupled to the proximal connector, wherein the blood is configured to flow through the extension tube, wherein in response to compression of the compressible portion, the extension tube is pinched and the housing is configured to dispense the blood from the extension tube distally through the distal end of the housing.

14. The instrument advancement device of claim 10, wherein the compressible portion is aligned with the slot.

15. An instrument advancement device, comprising:
a housing, comprising a proximal end, a distal end, and a slot;
an advancement element extending through the slot and configured to move linearly along the slot between a retracted position and an advanced position; and
an instrument comprising a first end and a second end, wherein in response to movement of the advancement element from the retracted position to the advanced position, the instrument is advanced beyond the distal end of the housing,
wherein the distal end of the housing comprises a slot and a slider configured to move along the slot, wherein in response to movement of the slider from a proximal end of the slot to a distal end of the slot, blood is dispensed distally through the distal end of the housing.

16. The instrument advancement device of claim 15, wherein the slider is coupled to a septum disposed within a chamber of the distal end of the housing, wherein the septum is configured to move with the slider.

17. The instrument advancement device of claim 16, wherein the chamber is configured to hold blood, wherein the septum extends across the chamber.

18. The instrument advancement device of claim 15, wherein the distal end of the housing comprises a distal connector, wherein the distal connector comprises the slider, the slot, and the septum.

19. The instrument advancement device of claim 18, wherein the distal connector comprises a pair of opposing lever arms, wherein the slider is disposed between the pair of opposing lever arms.

20. The instrument advancement device of claim 15, wherein the instrument comprises a guidewire.
